(19) United States
(12) Patent Application Publication
(10) Pub. No.: US 2026/0220944 A1
(43) Pub. Date: Jul. 30, 2026

(54) SUSPICIOUS PERSON DETECTION DEVICE, SUSPICIOUS PERSON DETECTION METHOD, AND RECORDING MEDIUM

(71) Applicant: NEC Corporation, Tokyo (JP)

(72) Inventors: Ryosuke SHIMIZU, Tokyo (JP); Masaaki KIMURA, Tokyo (JP); Ying XIN, Tokyo (JP); Hideki KOYANAGAWA, Tokyo (JP)

(73) Assignee: NEC Corporation, Tokyo (JP)

(21) Appl. No.: 19/145,265

(22) PCT Filed: Mar. 30, 2023
(86) PCT No.: PCT/JP2023/013071

§ 371 Date: Jul. 2, 2025

(51) Int. Cl. G06V 20/52; A61B 5/00; G06V 40/20; G08B 13/196

(52) U.S. Cl. CPC G06V 20/52; A61B 5/746; G06V 40/25; G08B 13/19602

(57) ABSTRACT

This suspicious person detection device includes: one or more memories storing instructions; and one or more processors configured to execute the instructions to: acquire a video of a person; acquire vital data about the person; determine the state of the person represented by the vital data; determine whether the situation of the person based on the video matches the situation anticipated from the state of the person represented by the vital data; and output an alert if it is determined that the situation of the person based on the video does not match the situation anticipated from the state of the person represented by the vital data.

### TECHNICAL FIELD

The present disclosure relates to a suspicious person detection device, a suspicious person detection method, and a recording medium.

### BACKGROUND ART

There is a technology for detecting a suspicious person who is a person who may perform an illegal act such as a crime and notifying the suspicious person. For example, there is a technology for detecting a specific behavior of a person, a combination of specific behaviors of the person, or the like, detecting a suspicious person, and issuing a notification. For example, there is a technology for detecting a suspicious person, by using a vibration pattern generated by a person or thermal image data of the person and issuing a notification.

PTL 1 describes a suspicious person notification system that calculates a stress index from vital data such as brain waves, a brain blood flow, pulse waves, a blood pressure, a respiratory rate, a body temperature, or an amount of perspiration, estimates that a person having a stress index higher than a reference value as a suspicious person, and issues a warning in a case where the suspicious person enters a specific area.

### CITATION LIST

### Patent Literature

PTL 1: JP 2018-037075 A

### SUMMARY OF INVENTION

### Technical Problem

However, the suspicious person notification system described in PTL 1 may estimate a person having a high stress index due to a reason other than an illegal act as the suspicious person.

An example of objects of the present disclosure is to provide a technology for more accurately detecting a suspicious person.

### Solution to Problem

A suspicious person detection device according to one mode of the present disclosure includes video acquisition means for acquiring a video of a person, vital data acquisition means for acquiring vital data of the person, first determination means for determining a state of the person indicated by the vital data, second determination means for determining whether a situation of the person based on the video matches a situation assumed from the state of the person indicated by the vital data, and output means for outputting an alert, in a case where it is determined that the situation of the person based on the video does not match the situation assumed from the state of the person indicated by the vital data.

A suspicious person detection method according to one mode of the present disclosure by a computer, includes acquiring a video of a person, acquiring vital data of the person, determining a state of the person indicated by the vital data, determining whether a situation of the person based on the video matches a situation assumed from the state of the person indicated by the vital data, and outputting an alert, in a case where it is determined that the situation of the person based on the video does not match the situation assumed from the state of the person indicated by the vital data.

A suspicious person detection program according to one mode of the present disclosure causes a computer to execute processing including acquiring a video of a person, acquiring vital data of the person, determining a state of the person indicated by the vital data, determining whether a situation of the person based on the video matches a situation assumed from the state of the person indicated by the vital data, and outputting an alert, in a case where it is determined that the situation of the person based on the video does not match the situation assumed from the state of the person indicated by the vital data.

Each program may be stored in a non-transitory computer-readable recording medium.

### Advantageous Effects of Invention

An example of an effect of the present disclosure is that a suspicious person can be more accurately detected.

### BRIEF DESCRIPTION OF DRAWINGS

FIG. 1 is a diagram illustrating an overview of a system including a suspicious person detection device.

FIG. 2 is a block diagram illustrating a functional configuration of the suspicious person detection device.

FIG. 3 is a diagram illustrating an example of an alert output screen.

FIG. 4 is a flowchart illustrating an operation of the suspicious person detection device.

FIG. 5 is a diagram illustrating a hardware configuration for achieving the suspicious person detection device according to the present disclosure by a computer and peripheral apparatuses thereof.

### EXAMPLE EMBODIMENT

### Example Embodiment

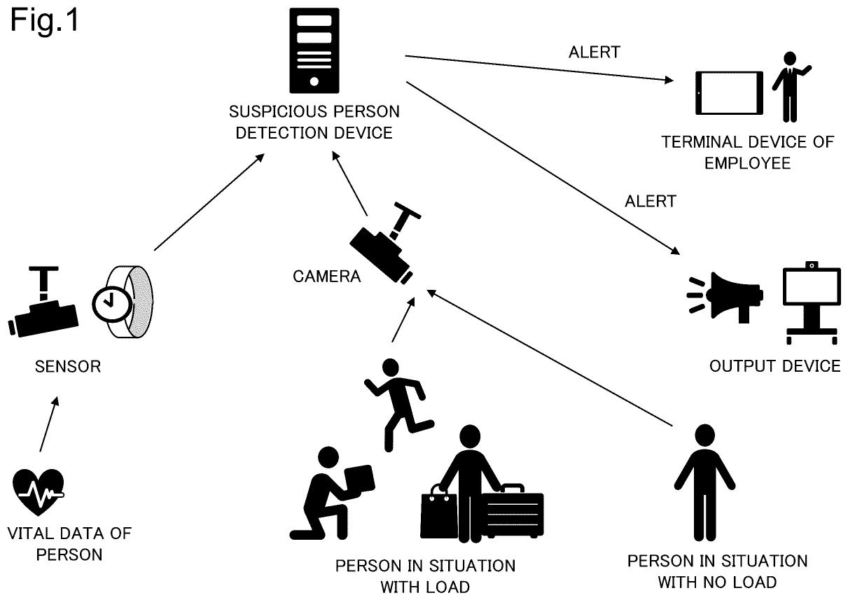

FIG. 1 is a diagram illustrating an overview of a system including a suspicious person detection device.

The suspicious person detection device is a device that detects a suspicious person by using vital data and a video of a person.

The suspicious person detection device is communicably connected to a sensor for acquiring the vital data of the person, a camera for acquiring the video of the person, and an output device that outputs an alert. Although the suspicious person detection device detects a suspicious person, for example, in a facility such as a store, the suspicious person detection device is not limited to this.

Here, the vital data may be at least one of physiological information of the person, information indicating an inner state of the person, and information indicating a mental state of the person. The vital data may be, for example, a heart rate, a respiratory rate, a body temperature, an opening degree of a pupil, or the like, which are the physiological information. For example, the vital data may be a numerical value of a stress value estimated by using various types of physiological information or an inner state or a mental state of the person such as a tension level or an aggressiveness.

The sensor for acquiring the vital data of the person is a sensor that measures the vital data of the person. The sensor for acquiring the vital data of the person may be, for example, a wearable terminal or a sensor attached to a cart or a basket used to carry products in a store. The sensor for acquiring the vital data of the person may be a camera that images the person. In this case, the vital data is extracted, by analyzing a change in a facial color, a movement of a face, a line-of-sight, blinks, a pupil diameter, a movement of an iris, an expression, or the like, included in a face video of the person imaged by the camera.

The camera for acquiring the video of the person is, for example, a monitoring camera. The camera for acquiring the video of the person may be a camera of a terminal device used for self-scan shopping in which a customer scans a product by oneself in a store. Note that, in a case where the sensor for acquiring the vital data of the person is a camera, the same camera may be used.

The output device that outputs the alert is a device that outputs an alert by displaying sound and a video. The output device may be, for example, a display or a speaker. Furthermore, the output device may be a terminal device.

Next, a configuration of a suspicious person detection device 100 according to an example embodiment will be described.

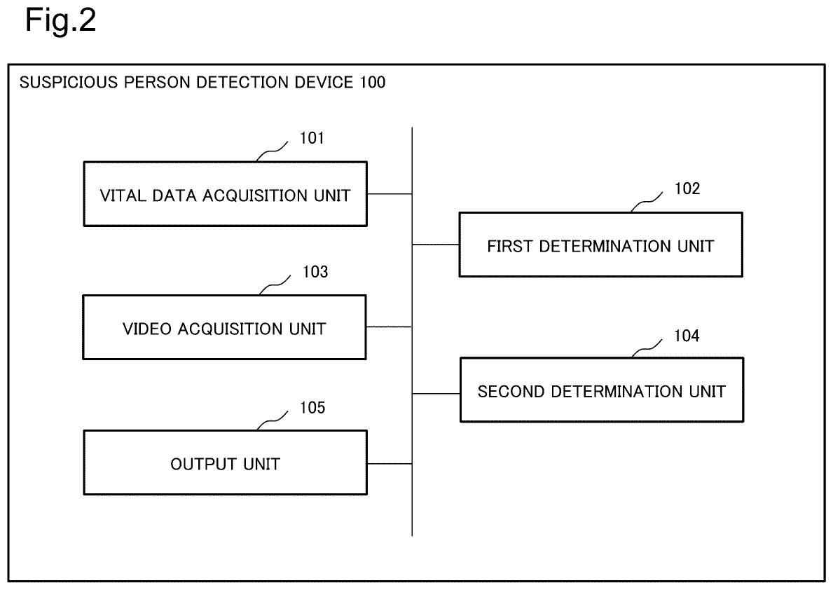

FIG. 2 is a block diagram illustrating the configuration of the suspicious person detection device 100 according to a first example embodiment. Referring to FIG. 2, the suspicious person detection device 100 includes a vital data acquisition unit 101, a first determination unit 102, a video acquisition unit 103, a second determination unit 104, and an output unit 105.

Next, the configuration of the suspicious person detection device 100 according to the first example embodiment will be described in detail.

In FIG. 2, the vital data acquisition unit 101 is an example of vital data acquisition means for acquiring vital data of a person. The vital data acquisition unit 101 may continuously acquire the vital data or may acquire the vital data at regular time intervals.

The vital data acquisition unit 101 acquires the vital data, for example, from the sensor that measures the vital data. The sensor that measures the vital data may be included, for example, in a wearable terminal, a communicable terminal device such as a smart cart used by a customer in a store. The vital data acquisition unit 101 may associate identification information of the sensor or the terminal device that acquires the vital data with the acquired vital data. The vital data acquisition unit 101 may associate identification information such as membership information of the person associated with the sensor or the terminal device that acquires the vital data, with the acquired vital data.

For example, the vital data acquisition unit 101 may acquire vital data based on a video. In this case, the vital data acquisition unit 101 may acquire the video from the camera and extract the vital data. For example, the vital data acquisition unit 101 may acquire the vital data, from another device that extracts vital data from a video. The vital data acquisition unit 101 may associate identification information of a terminal device including the camera that captures the video from which the vital data is extracted, with the extracted vital data. The vital data acquisition unit 101 may associate the identification information such as the membership information of the person associated with the terminal device including the camera that captures the video from which the vital data is extracted, with the acquired vital data. Moreover, in a case where the camera that captures the video from which the vital data is extracted is not related to each person, such as a monitoring camera, the vital data acquisition unit 101 may associate an identification number for identifying a person or the like with the acquired vital data.

The vital data acquisition unit 101 may acquire both of the vital data measured by the sensor and the vital data based on the video. For example, the vital data acquisition unit 101 may acquire vital data measured by a measurement sensor from a person who uses the measurement sensor and acquire vital data based on a video from a person who does not use the measurement sensor.

The first determination unit 102 is an example of first determination means for determining a state of the person indicated by the vital data. For example, the first determination unit 102 determines that the state of the person indicated by the vital data may lead to a state where the person performs an illegal act. In order to determine this state, for example, it is sufficient for the first determination unit 102 to determine whether the vital data satisfies a condition of vital data in a case where an illegal act is performed. The condition of the vital data in a case where the illegal act is performed may be a value of vital data in a case where the person performs the illegal act or the person is performing the illegal act or a change in the value of the vital data.

Here, the illegal act is a criminal act, a violent act, intimidation, or the like. For example, an illegal act in a store is shoplifting, burglary, or the like. Generally, in a case where an illegal act is performed and a case where an illegal act is to be performed, the person has a change in emotion such as an increase in the tension level or an increase in the stress. Such a change in emotion appears in the vital data. For example, the change in the vital data in a case where the illegal act is performed or the illegal act is to be performed is a change such as an increase in the heart rate, an increase in a skin temperature, an increase in the respiratory rate, or opening of the pupil. That is, the condition of the vital data in a case where the illegal act is performed may be that a numerical value indicated by the vital data exceeds a predetermined threshold or that a change amount of the numerical value indicated by the vital data exceeds the predetermined threshold. For example, the condition of the vital data in a case where the illegal act is performed may be that the change in the vital data is similar to the change in the vital data in a case where the illegal act is performed. Here, the similarity may be that a difference is equal to or less than a certain value or a similarity is larger than a threshold. The condition of the vital data in a case where the illegal act is performed is not limited to these examples and may be appropriately determined according to vital data to be used. The first determination unit 102 is only required to determine whether the vital data of the person satisfies the condition of the vital data in a case where the illegal act is performed.

The first determination unit 102 may make a determination, by using a plurality of types of vital data. For example, although the first determination unit 102 may determine whether the heart rate and the opening degree of the pupil satisfy the condition of the vital data in a case where the illegal act is performed, a combination of the pieces of vital data determined by the first determination unit 102 is not limited to this example.

The video acquisition unit 103 is an example of video acquisition means for acquiring a video of a person. The video of the person is a video used to determine a situation where a person is placed, by the second determination unit 104 to be described later. The video acquisition unit 103 acquires a video from a camera that images a person. As described above, the camera may be a monitoring camera, a camera of a terminal device used for scan shopping, or the like. A timing when the video acquisition unit 103 acquires the video of the person is not particularly limited. For example, in a case where the first determination unit 102 determines that the vital data satisfies the condition in a case where the illegal act is performed, the video acquisition unit 103 may acquire the video of the person.

For example, the video acquisition unit 103 acquires a video imaging a person whose vital data satisfies the condition of the vital data in a case where the illegal act is performed. For example, in a case of acquiring vital data measured by a measurement sensor included in a terminal device used by a person, the video acquisition unit 103 may specify a camera including a position indicated by position information of the terminal device within an imaging range, based on the position information of the terminal device including the measurement sensor and acquire a video imaged by the specified camera. In a case where the vital data acquisition unit 101 acquires the vital data based on the video, the video acquisition unit 103 may acquire the video used to acquire the vital data. For example, in a case where the vital data is extracted from the video acquired by the vital data acquisition unit 101, the video acquisition unit 103 may acquire the video used to extract the vital data. In addition, for example, in a case where the vital data acquisition unit 101 acquires vital data extracted from a video from another apparatus, the video acquisition unit 103 may acquire the video used to extract the vital data from the camera or the another apparatus.

The video acquisition unit 103 acquires a video at a vital data acquisition time point which is a time point when vital data is acquired from a person. For example, the video acquisition unit 103 acquires a video at a time point when the vital data that satisfies the condition of the vital data in a case where the illegal act is performed is acquired from the person. The time point when the vital data is acquired from the person is a time point of measurement if the vital data acquisition unit 101 acquires the vital data from the measurement sensor and a time point when the video is imaged, if the vital data acquisition unit 101 acquires the vital data based on the video.

For example, the video acquisition unit 103 may acquire a video at a predetermined time before the time point when the vital data is acquired from the person. This is because, at the time point when the vital data that satisfies the condition of the vital data in a case where the illegal act is performed is acquired from the person, a situation such as a behavior of the person in a period before that time point may affect the vital data of the person. For example, it is considered that a heart rate of a person having a high heart rate at a certain time point increases due to running before the certain time point and is still high in a case where the person is walking at the certain time point. It is sufficient that the predetermined time before the time point when the vital data is acquired from the person, when the video acquisition unit 103 acquires the video be a time at which the vital data at the vital data acquisition time point is affected and be appropriately set.

The second determination unit 104 is an example of second determination means for determining whether a situation of a person based on a video matches a situation assumed from the state of the person indicated by the vital data. For example, in a case where the vital data is determined to satisfy the condition of the vital data in a case where the illegal act is performed, in the determination by the first determination unit 102, the second determination unit 104 determines whether the situation of the person based on the video of the person acquired by the video acquisition unit 103 is a situation that matches the state of the person indicated by the vital data.

The situation of the person is at least one of a behavior of the person, a movement of the person, and an environment around the person. For example, the second determination unit 104 may detect the movement of the person and the movement of the person, from the video of the person, by a known method. The environment around the person may be, for example, a movement or a behavior of another person existing around the person, detected from the video. In this case, for example, the second determination unit 104 may detect the movement or the behavior of the another person existing in a present distance range from the person, from the video of the person, by a known method. For example, the environment around the person may be detected from sound detected around the person. In this case, for example, the second determination unit 104 may detect sound included in the video of the person, by a known method.

Even if the vital data satisfies the condition of the vital data in a case where the illegal act is performed, there is a case where the vital data satisfies the condition of the vital data in a case where the illegal act is performed, due to another factor other than the illegal act. Therefore, the second determination unit 104 makes a determination by using the video, regarding the situation of the person whose the vital data that satisfies the condition of the vital data in a case where the illegal act is performed is acquired. Therefore, the second determination unit 104 determines whether the situation of the person based on the video of the person is the situation that matches the state of the person indicated by the vital data. That is, in a case where there is a situation where tension or an emotional state of the person indicated by the vital data serves as a factor, the second determination unit 104 determines that the situation of the person based on the video of the person whose the vital data that satisfies the condition of the vital data in a case where the illegal act is performed is acquired is the situation that matches the situation assumed from the state of the person indicated by the vital data. In a case where such a determination is made, the person is not treated as the suspicious person. On the other hand, in a case where the vital data satisfies the condition of the vital data in a case where the illegal act is performed and it is not determined that there is a situation to be the factor, the second determination unit 104 determines that the situation of the person based on the video of the person whose the vital data that satisfies the condition of the vital data in a case where the illegal act is performed is acquired is a situation that does not match the situation assumed from the state of the person indicated by the vital data. In a case where such a determination is made, the person may be treated as the suspicious person.

Here, the another factor other than the illegal act with which the vital data of the person satisfies the condition of the vital data in a case where the illegal act is performed is, for example, a situation where a physical load is applied to the person or a situation where a mental load is applied due to an external factor.

The situation where the physical load is applied to the person is, for example, a situation where a person is exercising. For example, the situation where the physical load is applied to the person is that the person is running. In this case, the second determination unit 104 determines that the person is running, based on the video of the person. For example, the second determination unit 104 may determine that the person is running in accordance of a moving speed of the person specified from the video or a posture of the person specified from the video. In addition, for example, the situation where the physical load is applied to the person is that the person carries a heavy object. In this case, the second determination unit 104 may determine that the person carries the heavy object from a size of a baggage of the person specified from the video.

The situation where the mental load is applied to the person due to the external factor is, for example, a situation where the person is surprised due to the external factor or a situation where the person feels uncomfortable. For example, the situation where the mental load is applied to the person due to the external factor is that the person is surprised at loud sound. In this case, the second determination unit 104 determines loud sound of which a value representing a magnitude of sound around the person is equal to or more than a threshold is made, based on the video of the person including the sound. In this case, the second determination unit 104 may determine that loud sound is suddenly made around the person. This is because, in a situation where the loud sound is continuously made, the person gets used to the loud sound and is less likely to be surprised. In addition, for example, the situation where the mental load is applied to the person due to the external factor is that the person collides with another person or a baggage of the another person. In this case, the second determination unit 104 may determine that the person comes into contact with the another person or the baggage of the another person, based on the video of the person.

In a case where the second determination unit 104 determines that the situation of the person based on the video of the person at the vital data acquisition time point is the situation that does not match the situation assumed from the state of the person indicated by the vital data, the second determination unit 104 may make a similar determination on the situation based on the video of the person at the predetermined time before the time point when the vital data is acquired from the person. In this case, in response to the determination that the situation of the person based on the video at the vital data acquisition time point does not match the situation assumed from the state of the person indicated by the vital data, the video acquisition unit 103 may acquire the video at the predetermined time before the vital data acquisition time point.

This indicates that, as described above, because the state of the person indicated by the vital data may be affected by the situation at the time before the vital data acquisition time point, the second determination unit 104 makes a determination on the situation based on the video of the person at the predetermined time before the time point when the vital data is acquired from the person. However, if the second determination unit 104 makes a determination on the situation at the time before the vital data acquisition time point every time, a processing load increases. On the other hand, as described above, in a case where the situation of the person based on the video of the person at the vital data acquisition time point is determined as the situation that does not match the situation assumed from the state of the person indicated by the vital data, the second determination unit 104 can reduce the processing load, by making a determination on the situation of the person based on the video of the person at the predetermined time before the vital data acquisition time point.

The output unit 105 is an example of output means for outputting an alert, in a case where the second determination unit 104 determines that the situation of the person does not match the situation assumed from the state of the person indicated by the vital data. The output unit 105 may output the alert to at least one of a display, a speaker, or a terminal device.

First, an output determination to which the output unit 105 outputs the alert will be described.

The output unit 105 may output an alert to a person (hereinafter, described as a suspicious person) whose vital data is determined, by the first determination unit 102, to satisfy the condition of the vital data in a case where the illegal act is performed and whose situation is determined, by the second determination unit 104, not to match the situation assumed from the state of the person indicated by the vital data. In this case, for example, the output unit 105 may output the alert to an output device near a position of the person from the video or information of the camera that has imaged the video. The information of the camera may be position information of the camera, information indicating an imaging range of the camera, or identification information of the camera associated with these pieces of information. For example, the output unit 105 may output the alert to the terminal device used to acquire the vital data video.

The output unit 105 may output the alert to a person such as an employee other than the suspicious person. In this case, the output unit 105 is only required to output the alert to a predetermined terminal device or the like. For example, the output unit 105 may output the alert, to a terminal device used by an employee of a facility where the person stays.

In a case where the suspicious person is the employee, the output unit 105 may output the alert to a terminal device used by an administrator of the facility. The suspicious person may be identified as the employee in accordance with that clothes of the person based on the video is a uniform of the employee, that the person has a company ID based on the video, information in which a terminal device that has acquired the vital data is associated with identification information of the employee in advance, or the like.

Next, content of the alert output by the output unit 105 will be described.

The alert to the suspicious person is issued, for example, in such a way as to prevent the suspicious person from performing an illegal act. For example, the alert to the suspicious person may be sound and a video that calls attention to the illegal act, sound that attracts attention from surrounding people, or the like. Due to the sound that attracts attention from the surrounding people, it is considered that the suspicious person is less likely to perform the illegal act because the suspicious person attracts attention from the surrounding people. Content indicated by the sound that attracts attention from the surrounding people is not particularly limited, and may be content calling attention to the illegal act, content of making an advertisement related to a store or the like, or content of asking whether an action such as “May I help you?” is needed.

The alert to the person other than the suspicious person is issued, for example, to cause a person other than the suspicious person such as the employee to perform a behavior for preventing the suspicious person from performing the illegal act. The alert to the person other than the suspicious person may be issued to specify the suspicious person, in a case where the illegal act is found. For example, the alert to the person other than the suspicious person includes content that can specify the suspicious person. The content that can specify the suspicious person may be at least one of a current position of the suspicious person, a position of the suspicious person at the vital data acquisition time point, and a video of the suspicious person.

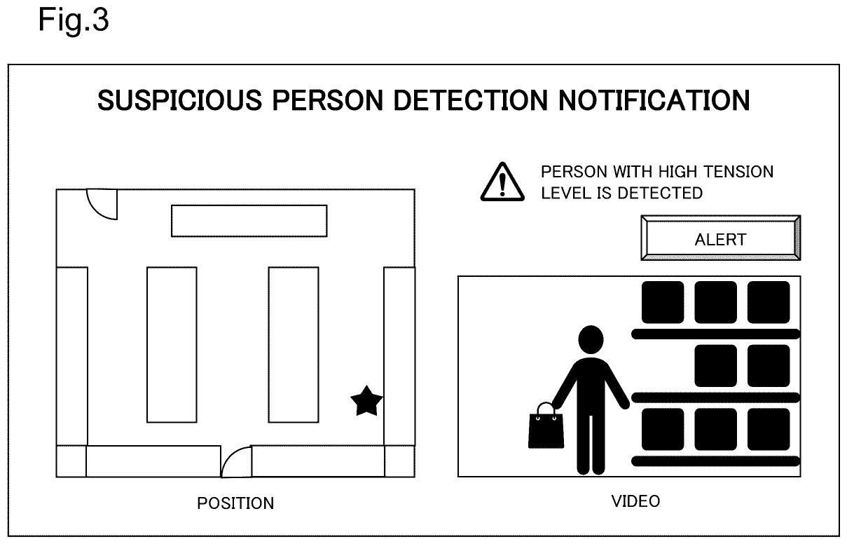

FIG. 3 is an output screen example of the alert to the employee. In FIG. 3, an output screen of the alert includes detection of the suspicious person, a position of the suspicious person in the store, and the video of the suspicious person. In addition, in FIG. 3, the output screen of the alert includes an “alert” button. For example, in a case where the “alert” button is selected on the output screen of the alert to the employee, the output unit 105 may output an alert for notifying another employee or a security guard of that the suspicious person is detected. For example, in a case where the button described above is selected, the output unit 105 may output an alert to the person detected as the suspicious person. In this way, it is considered that there is a case where it is possible to prevent a person such as a customer from being erroneously alerted or to prevent a person with increasing aggressiveness from being stimulated, by determining whether to issue an alert based on the determination by the employee.

Next, a case where the situation of the person based on the video is determined to match the situation assumed from the state of the person indicated by the vital data will be described.

For example, in a case where the situation of the person based on the video is determined to match the situation assumed from the state of the person indicated by the vital data, the output unit 105 does not issue an alert. For example, in a case where the situation of the person based on the video is determined to match the situation assumed from the state of the person indicated by the vital data, the output unit 105 may output that the person is not the suspicious person. For example, the output unit 105 may superimpose and display whether the situation of the person based on the video matches the situation assumed from the state of the person indicated by the vital data on each person identifiably displayed in the video.

For example, the output unit 105 may output information regarding the person whose vital data satisfies the condition of the vital data in a case where the illegal act is performed. For example, in a case where the situation of the person based on the video is determined to match the situation assumed from the state of the person indicated by the vital data, the output unit 105 may output that the situation of the person whose vital data satisfies the condition of the vital data in a case where the illegal act is performed matches the situation assumed from the state of the person indicated by the vital data. For example, the output unit 105 may output the situation of the person based on the video, such as the person whose vital data satisfies the condition of the vital data in a case where the illegal act is performed is running.

In a case where it is not possible for the vital data acquisition unit 101 to acquire vital data of an employee who is wearing the measurement sensor for a predetermined time or longer, the output unit 105 may output the alert. As a result, the illegal act can be prevented, even in a case where an employee who knows that the vital data is used to detect the suspicious person intentionally makes it impossible to acquire the vital data in such a way as to perform the illegal act without being detected as the suspicious person.

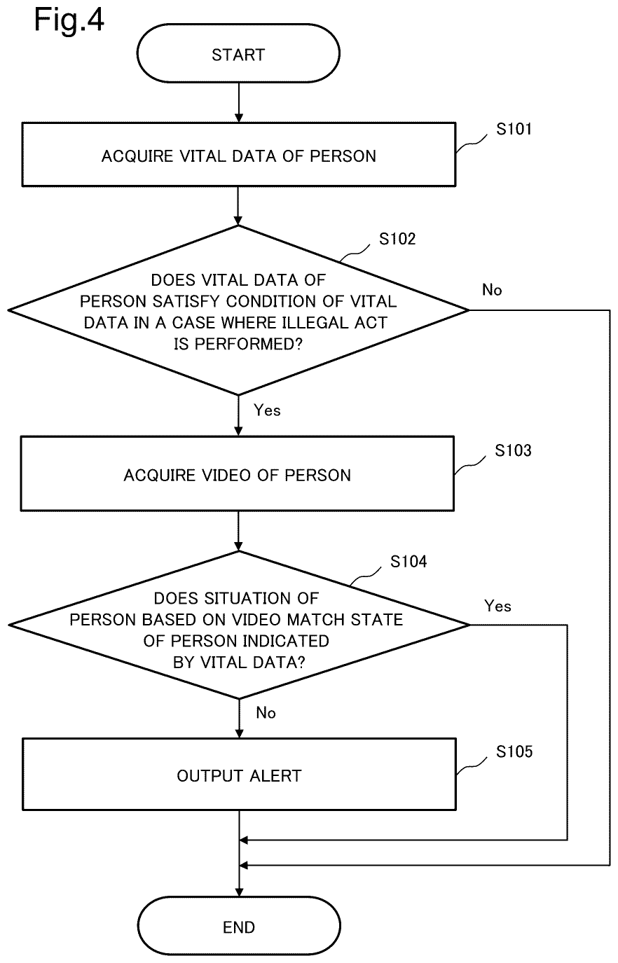

An operation of the suspicious person detection device 100 configured as described above will be described with reference to the flowchart in FIG. 4.

As illustrated in FIG. 4, first, the vital data acquisition unit 101 acquires the vital data of the person (step S101). The vital data may be constantly acquired from each person included in the facility.

Next, the first determination unit 102 determines whether the vital data of the person acquired in step S101 satisfies the condition of the vital data in a case where the illegal act is performed (step S102). In a case where it is not determined in step S102 that the vital data of the person satisfies the condition of the vital data in a case where the illegal act is performed (step S102: No), the suspicious person detection device 100 ends the processing.

In a case where it is determined that the vital data of the person satisfies the condition of the vital data in a case where the illegal act is performed in the determination in step S102 (step S102: Yes), the video acquisition unit 103 acquires the video of the person (step S103).

Next, the second determination unit 104 determines whether the situation of the person based on the video of the person acquired in step S103 matches the situation assumed from the state of the person indicated by the vital data acquired in step S101 (step S104). In a case where the situation of the person based on the video is determined to match the situation assumed from the state of the person indicated by the vital data in step S104 (step S104: Yes), the suspicious person detection device 100 may end the processing.

In a case where it is determined that the situation of the person based on the video does not match the situation assumed from the state of the person indicated by the vital data (step S104: No), the output unit 105 outputs the alert.

As described above, the suspicious person detection device 100 ends the series of processing. The series of processing may be repeatedly performed. An order of each processing may be appropriately changed. For example, the step for acquiring the video of the person in step S103 may be performed before or simultaneously with step S101.

In the suspicious person detection device 100 according to the present example embodiment described above, the first determination unit 102 determines the state of the person indicated by the vital data, and the second determination unit 104 determines whether the situation of the person based on the video of the person matches the situation that matches the situation assumed from the state of the person indicated by the vital data. Then, in a case where it is determined that the situation of the person does not match the situation assumed from the state of the person indicated by the vital data, the output unit 105 outputs the alert.

As a result, because the suspicious person detection device 100 according to the present example embodiment does not issue the alert in a case where the vital data satisfies the condition under which the illegal act is performed due to the factor other than the illegal act, as compared with a case where the suspicious person is detected only based on the vital data, the suspicious person detection device 100 can more accurately detect the suspicious person.

### [Hardware Configuration]

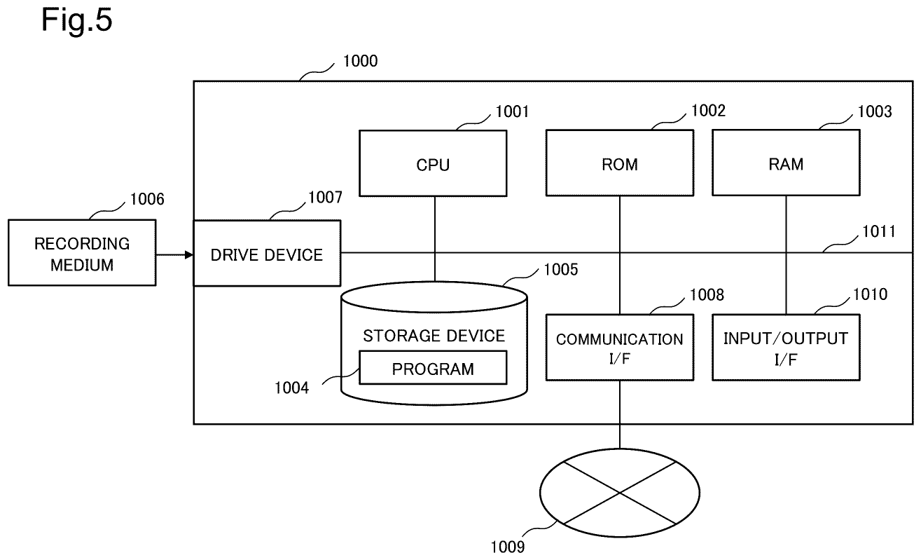

Some or all of the components of each apparatus or system in the example embodiment of the present disclosure described above are achieved by, for example, any combination of an information processing apparatus 1000 and a program as illustrated in FIG. 5. As an example, the information processing apparatus 1000 includes the following configurations.
Central processing unit (CPU) 1001
Read only memory (ROM) 1002
Random access memory (RAM) 1003
Program 1004 loaded into RAM 1003
Storage device 1005 storing program 1004
Drive device 1007 for reading and writing recording medium 1006
Communication I/F 1008 connected to communication network 1009
Input/output I/F 1010 for inputting/outputting data
Bus 1011 connecting each component

The I/F is an abbreviation of Interface.

Each component of each apparatus or system in each example embodiment is achieved by the CPU 1001 by acquiring and executing a program for achieving these functions. The program for achieving the function of each component of each apparatus is stored in the storage device 1005 or the RAM 1003 in advance, for example, and is read by the CPU 1001 as necessary. The program 1004 may be supplied to the CPU 1001 via a communication network, or may be stored in advance in the recording medium 1006, and the drive device 1007 may read the program and supply the program to the CPU 1001.

There are various modifications of the method of achieving each apparatus. For example, each apparatus or system may be achieved by any combination of the information processing apparatus 1000 and the program separate for each component. A plurality of components included in each apparatus may be achieved by any combination of one information processing apparatus 1000 and a program.

Some or all of the components of each apparatus or system are achieved by a general-purpose or dedicated circuit (circuitry) including a processor or the like, or a combination thereof. The circuit is, for example, a CPU, a graphics processing unit (GPU), a field programmable gate array (FPGA), or a large scale integration (LSI) for artificial intelligence (AI) processing. These components may be configured by a single chip, or may be configured by a plurality of chips connected to each other via a bus. Some or all of the components of each apparatus may be achieved by a combination of the above-described circuit and the like and a program.

In a case where some or all of the components of each apparatus or system are achieved by a plurality of information processing apparatuses, circuits, and the like, the plurality of information processing apparatuses, circuits, and the like may be arranged in a centralized manner or in a distributed manner. For example, the information processing apparatuses, the circuits, or the like may be achieved in a configuration where each of the components is connected via a communication network, such as a client-server system or a cloud computing system.

Although the present invention has been described with reference to the example embodiment, the present invention is not limited to the above example embodiment. It will be understood by those of ordinary skill in the art that various changes in form and details may be made therein without departing from the spirit and scope of the present invention as defined by the claims.

Although the plurality of operations is described in order in the form of a flowchart, the order of description does not limit the order of executing the plurality of operations. Therefore, when each example embodiment is implemented, the order of the plurality of operations may be changed within a range that does not interfere with the content.

Some or all of the above example embodiments may be described as the following Supplementary Notes, but are not limited to the following.

### [Supplementary Note 1]

A suspicious person detection device including:
video acquisition means for acquiring a video of a person;
vital data acquisition means for acquiring vital data of the person;
first determination means for determining a state of the person indicated by the vital data;
second determination means for determining whether a situation of the person based on the video matches a situation assumed from the state of the person indicated by the vital data; and
output means for outputting an alert, in a case where it is determined that the situation of the person based on the video does not match the situation assumed from the state of the person indicated by the vital data.

### [Supplementary Note 2]

The suspicious person detection device according to supplementary note 1, in which
the first determination means determines that the state of the person indicated by the vital data is a state where there is a possibility that an illegal act is performed.

### [Supplementary Note 3]

The suspicious person detection device according to supplementary note 1 or 2, in which
in a case where the situation is a situation where a physical load is applied to the person, the second determination means determines that the situation is a situation that matches the situation assumed from the state of the person indicated by the vital data.

### [Supplementary Note 4]

The suspicious person detection device according to supplementary note 3, in which
the situation where the physical load is applied to the person is a situation where the person is running.

### [Supplementary Note 5]

The suspicious person detection device according to supplementary
the situation where the physical load is applied to the person is a situation where the person carries a large baggage. note 3 or 4, in which

### [Supplementary Note 6]

The suspicious person detection device according to any one of supplementary notes 1 to 5, in which
in a case where the situation is a situation where an external factor applies a mental load to the person, the second determination means determines that the situation is the situation that matches the situation assumed from the state of the person indicated by the vital data.

### [Supplementary Note 7]

The suspicious person detection device according to supplementary note 6, in which
the situation where the external factor applies the mental load to the person is a situation where loud sound is made near the person.

### [Supplementary Note 8]

The suspicious person detection device according to supplementary note 6 or 7, in which
the situation where the external factor applies the mental load to the person is a situation where the person collides with another person.

### [Supplementary Note 9]

The suspicious person detection device according to any one of supplementary notes 1 to 8, in which
the video is a video at an acquisition time point which is a time point when the vital data is acquired from the person.

### [Supplementary Note 10]

The suspicious person detection device according to supplementary note 9, in which
in a case where it is determined that a situation of the person based on the video at the acquisition time point does not match the situation of the person assumed from the state of the person indicated by the vital data, the video acquisition means acquires a video at a predetermined time before the acquisition time point, and
the second determination means determines whether a situation of the person based on the video at the predetermined time before the acquisition time point is the situation that matches the situation of the person assumed from the state of the person indicated by the vital data.

### [Supplementary Note 11]

The suspicious person detection device according to any one of supplementary notes 1 to 10, in which
the output means outputs an alert to an output device near the person.

### [Supplementary Note 12]

The suspicious person detection device according to any one of supplementary notes 1 to 11, in which
the output means outputs an alert to a terminal device used by an employee of a facility where the person stays.

### [Supplementary Note 13]

The suspicious person detection device according to supplementary note 12, in which
in a case where the person is the employee of the facility, the output means outputs an alert to a terminal device used by an administrator of the facility.

### [Supplementary Note 14]

The suspicious person detection device according to supplementary note 12 or 13, in which
the output means outputs an alert including the vital data and the video.

### [Supplementary Note 15]

The suspicious person detection device according to any one of supplementary notes 1 to 14, in which
in a case where the person is the employee of the facility and it is not possible to acquire vital data by non-video for a predetermined time or longer, the output means outputs an alert.

### [Supplementary Note 16]

A suspicious person detection method by a computer, including:
acquiring a video of a person;
acquiring vital data of the person;
determining a state of the person indicated by the vital data;
determining whether a situation of the person based on the video matches a situation assumed from the state of the person indicated by the vital data; and
outputting an alert, in a case where it is determined that the situation of the person based on the video does not match the situation assumed from the state of the person indicated by the vital data.

### [Supplementary Note 17]

A recording medium recording a program for causing a computer to execute processing including:
acquiring a video of a person;
acquiring vital data of the person;
determining a state of the person indicated by the vital data;
determining whether a situation of the person based on the video matches a situation assumed from the state of the person indicated by the vital data; and
outputting an alert, in a case where it is determined that the situation of the person based on the video does not match the situation assumed from the state of the person indicated by the vital data.

### REFERENCE SIGNS LIST

100 suspicious person detection device
101 vital data acquisition unit
102 first determination unit
103 video acquisition unit
104 second determination unit
105 output unit
1000 information processing apparatus
1001 CPU
1002 ROM
1003 RAM
1004 program
1005 storage device
1006 recording medium
1007 drive device
1008 communication I/F
1009 communication network
1010 input/output I/F
1011 bus

What is claimed is:

1. A suspicious person detection device comprising:
one or more memories storing instructions; and
one or more processors configured to execute the instructions to:
acquire a video of a person;
acquire vital data of the person;
determine a state of the person indicated by the vital data;
determine whether a situation of the person based on the video matches a situation assumed from the state of the person indicated by the vital data; and
output an alert, in a case where it is determined that the situation of the person based on the video does not match the situation assumed from the state of the person indicated by the vital data.

2. The suspicious person detection device according to claim 1, wherein the one or more processors is configured to execute the instructions to:
determine that the state of the person indicated by the vital data is a state where there is a possibility that an illegal act is performed.

3. The suspicious person detection device according to claim 1, wherein the one or more processors is configured to execute the instructions to:
in a case where the situation of the person based on the video is a situation where a physical load is applied to the person, determine that the situation of the person based on the video is a situation that matches the situation assumed from the state of the person indicated by the vital data.

4. The suspicious person detection device according to claim 3, wherein
the situation where the physical load is applied to the person is a situation where the person is running.

5. The suspicious person detection device according to claim 3, wherein
the situation where the physical load is applied to the person is a situation where the person carries a large baggage.

6. The suspicious person detection device according to claim 1, wherein the one or more processors is configured to execute the instructions to:
in a case where the situation of the person based on the video is a situation where an external factor applies a mental load to the person, determine that the situation of the person based on the video is the situation that matches the situation assumed from the state of the person indicated by the vital data.

7. The suspicious person detection device according to claim 6, wherein
the situation where the external factor applies the mental load to the person is a situation where loud sound is made near the person.

8. The suspicious person detection device according to claim 6, wherein
the situation where the external factor applies the mental load to the person is a situation where the person collides with another person.

9. The suspicious person detection device according to claim 1, wherein
the video is a video at an acquisition time point which is a time point when the vital data is acquired from the person.

10. The suspicious person detection device according to claim 9, wherein the one or more processors is configured to execute the instructions to:
in a case where it is determined that a situation of the person based on the video at the acquisition time point does not match the situation of the person assumed from the state of the person indicated by the vital data, acquire a video at a predetermined time before the acquisition time point, and
determine whether a situation of the person based on the video at the predetermined time before the acquisition time point is the situation that matches the situation of the person assumed from the state of the person indicated by the vital data.

11. The suspicious person detection device according to claim 1, wherein the one or more processors is configured to execute the instructions to:
output the alert to an output device near the person.

12. The suspicious person detection device according to claim 1, wherein the one or more processors is configured to execute the instructions to:
output the alert to a terminal device used by an employee of a facility where the person stays.

13. The suspicious person detection device according to claim 12, wherein the one or more processors is configured to execute the instructions to:
in a case where the person is the employee of the facility, output the alert to a terminal device used by an administrator of the facility.

14. The suspicious person detection device according to claim 12, wherein the one or more processors is configured to execute the instructions to:
output the alert including the vital data and the video.

15. The suspicious person detection device according to claim 1, wherein the one or more processors is configured to execute the instructions to:
in a case where the person is the employee of the facility and it is not possible to acquire vital data by non-video for a predetermined time or longer, output the alert.

16. A suspicious person detection method by a computer, comprising:
acquiring a video of a person;
acquiring vital data of the person;
determining a state of the person indicated by the vital data;
determining whether a situation of the person based on the video matches a situation assumed from the state of the person indicated by the vital data; and
outputting an alert, in a case where it is determined that the situation of the person based on the video does not match the situation assumed from the state of the person indicated by the vital data.

17. A non-transitory recording medium recording a program for causing a computer to execute processing comprising:
acquiring a video of a person;
acquiring vital data of the person;
determining a state of the person indicated by the vital data;
determining whether a situation of the person based on the video matches a situation assumed from the state of the person indicated by the vital data; and
outputting an alert, in a case where it is determined that the situation of the person based on the video does not match the situation assumed from the state of the person indicated by the vital data.
